(19) United States
(12) Patent Application Publication
(10) Pub. No.: US 2006/0235374 A1
(43) Pub. Date: Oct. 19, 2006

(54) In-ice detection for cryotherapy applications

(76) Inventor: William R. Mandel, Eden Prairie, MN (US)

Correspondence Address: AMS RESEARCH CORPORATION, 10700 BREN ROAD WEST, MINNETONKA, MN, 55343, US

(21) Appl. No.: 11/250,674

(22) Filed: Oct. 14, 2005

Related U.S. Application Data
(60) Provisional application No. 60/671,607, filed on Apr. 15, 2005.

(51) Int. Cl. A61B 18/02

(52) U.S. Cl. 606/20; 606/21

(57) ABSTRACT

A method and apparatus for heating a cryotherapy probe to maintain an elevated temperature, while evaluating heat on-time and heat off-time to determine whether the probe is within an existing ice ball. Indication that the probe is not within the existing ice ball signals the user that the formation of a second ice ball can begin.

### CLAIM TO PRIORITY

This application claims priority to U.S. Provisional Patent Application No. 60/671,607, filed on Apr. 15, 2005, and entitled “In-Ice Detection for Cryotherapy Applications.” The identified provisional patent application is hereby incorporated by reference.

### FIELD OF THE INVENTION

This invention is in the field of probes, and other equipment, which are capable of freezing biological tissue to achieve modification of the frozen tissue for therapeutic purposes, referred to herein as cryotherapy.

### BACKGROUND OF THE INVENTION

In the field of cryotherapy, it is typical to place a cryoprobe at a desired location adjacent to or within biological tissue, and then to freeze the biological tissue at that location by cooling of the probe. This is usually done to ultimately cause sloughing off of the tissue, to remedy an undesirable condition of that tissue. A freezing operation at a selected location is said to form an “ice ball” within the selected biological tissue, but it should be understood that the shape of the frozen tissue is seldom a perfect sphere, and that the material which becomes frozen within the “ice ball” often includes several types of tissue or even fluids. Often, it is desired to freeze tissue at more than one specific location within a treatment zone or organ, in order to necrotize a larger mass of the tissue than is possible with one freeze, or in order to achieve a desired shape of necrotized tissue. In this case, two or more ice balls may be formed in the tissue by successive applications of freezing with the probe, at two or more locations within the treatment zone.

For instance, it is known to freeze endometrial tissue within a uterus to treat endometriosis. Because the uterus is shaped with a somewhat flattened cross section, it is usually desirable to freeze a first ice ball in the tissue near one extremity of the uterus, then remove the probe from the first ice ball and freeze a second ice ball near the other extremity of the uterus. After removal of the probe from the first ice ball, it is usually desired to place the probe in a location that is at least not completely within the first ice ball, before beginning formation of the second ice ball. This is because, if the probe is reinserted into the first ice ball and freezing is resumed, the probe will only re-freeze the first ice ball and continue lowering its temperature and not achieve the desired coverage. This is not conducive to forming a second ice ball.

It should be understood that the typical tissue “ice ball” will not necessarily have a discrete outer surface or edge, being basically a mass of frozen tissue within an unfrozen mass of the same tissue. The ice ball may have a temperature of minus 90 degrees C. at its center, with a temperature gradient toward the outer portion of the ice ball, where a temperature of minus 20 degrees C. may be found. As the terms are used herein, if the probe is re-positioned to the approximate edge of the ice ball, that position is not considered to be fully within the ice ball, and it is considered to be an acceptable location for forming the second ice ball.

It is currently typical to use ultrasonic imaging to determine that probe placement is acceptable for forming of the second ice ball. However, this method is difficult to perform and it does not yield a reliable result. Often, if the probe is re-positioned within the first ice ball and freezing is commenced, ultrasonic imaging does not show that this has happened until the user notices that the ultrasonic imaging shows that a second ice ball is not being formed. This requires another release and re-positioning of the probe, resulting in a further time delay. It is desirable to have a more reliable means of ensuring that the re-positioned probe is at least not fully within the first ice ball, before the second freezing step is commenced.

### SUMMARY OF THE INVENTION

The present invention is a method and apparatus for determining when the re-positioned probe is at least not fully within the first ice ball the first ice ball by heating it with on-and-off cycles of a heating element. Successful placement of the probe in a second location which is not within the first ice ball is accomplished by monitoring the on-and-off cycles of the heating element. As the heater maintains the probe at the target temperature, the heater turns on when the probe temperature reaches the low end of a range, and then the heater turns off when the probe temperature reaches the target temperature. So, the probe temperature cycles between this lower limit of the range and the target temperature.

It has been found that, if the probe is re-positioned within the first ice ball, the heater on-time is longer, relative to the heater off-time, than when the probe is re-positioned outside of, or on the periphery of, the first i includes a processor which calculates the heater duty cycle for each of these heating cycles. Calculation of the duty cycle value compares the duration of the heater on-time to the duration of the heater off-time, or to the total of on-time and off-time, for the given cycle. Several duty cycles can be averaged, for more reliable results.

When the heater duty cycle value is above a selected setpoint, the processor determines that the probe is within the first ice ball. When the heater duty cycle is below this selected setpoint, the processor determines that the probe is at least not fully within the first ice ball. The setpoint for a given probe is selected as the duty cycle value that would occur if the probe were positioned at the periphery of the first ice ball. When the duty cycle value is calculated to be above the setpoint, the processor gives the user an indication that the probe is in ice and that freezing should not be commenced.

When the duty cycle value is calculated to be below the setpoint, the processor gives the user an indication that the probe is not in ice and that freezing of the second ice ball can commence. A time-out threshold can be selected, to insure that the probe surroundings do not have time to reach an equilibrium temperature, since temperature equilibrium around the probe would result in a very long on-time or a very long off-time. When the selected time-out threshold is reached, the processor terminates calculation of duty cycles until the probe is re-positioned.

One method of the present invention is directed to placing an instrument in a treatment zone having an existing ice ball therein. The method includes the following steps: (a) placing an instrument in a desired location within the treatment zone; (b) allowing the instrument to automatically pursue a desired temperature while within the treatment zone; (c) monitoring a temperature-based parameter of the instrument while it is within the treatment zone and while it is pursuing the desired temperature; and (d) indicating the location of the instrument relative to the existing ice ball based on the monitored temperature-based parameter of the instrument.

Another method of the present invention is directed to a method of placing a cryotherapy probe. The method includes the following steps: (a) placing a cryotherapy probe in a first location within a treatment zone; (b) cooling the probe to form an ice ball in the first location such that the ice ball has a temperature colder than the ambient temperature of the treatment zone; (c) raising the temperature of the probe sufficiently to melt ice immediately adjacent the probe so as to release the probe from the ice ball; (d) withdrawing the probe from the ice ball and placing it in a second location in the treatment zone; (e) allowing the probe to automatically pursue a desired temperature while within the second location; (f) measuring a temperature-based parameter of the probe while within the second location and while pursuing the desired temperature; and (g) evaluating relative the measured temperature-based parameter whether the probe is within the ice ball.

One preferred embodiment of the present invention is directed to a cryotherapy apparatus. The cryotherapy apparatus generally includes a cryotherapy probe, a sensor and a processor. The cryotherapy probe operates to automatically pursue a desired temperature and produces a temperature based parameter. The sensor monitors the temperature-based parameter and produces a signal representative of the temperature-based parameter. The processor receives the signal from the whether the cryotherapy probe is within or proximate an existing ice ball based on the signal from the sensor.

The novel features of this invention, as well as the invention itself, will be best understood from the attached drawings, taken along with the following description, in which similar reference characters refer to similar parts, and in which:

### DESCRIPTION OF THE DRAWINGS

FIG. 1 is a diagram of an in-ice detector of the present invention for cryotherapy applications.

FIG. 2 provides a graphical depiction of the relationship between heater on/off states and probe temperature when the probe is in heating mode.

FIG. 3 depicts various second locations of the probe proximate an existing ice ball within a treatment zone of a patient.

### DETAILED DESCRIPTION OF THE PREFERRED EMBODIMENTS

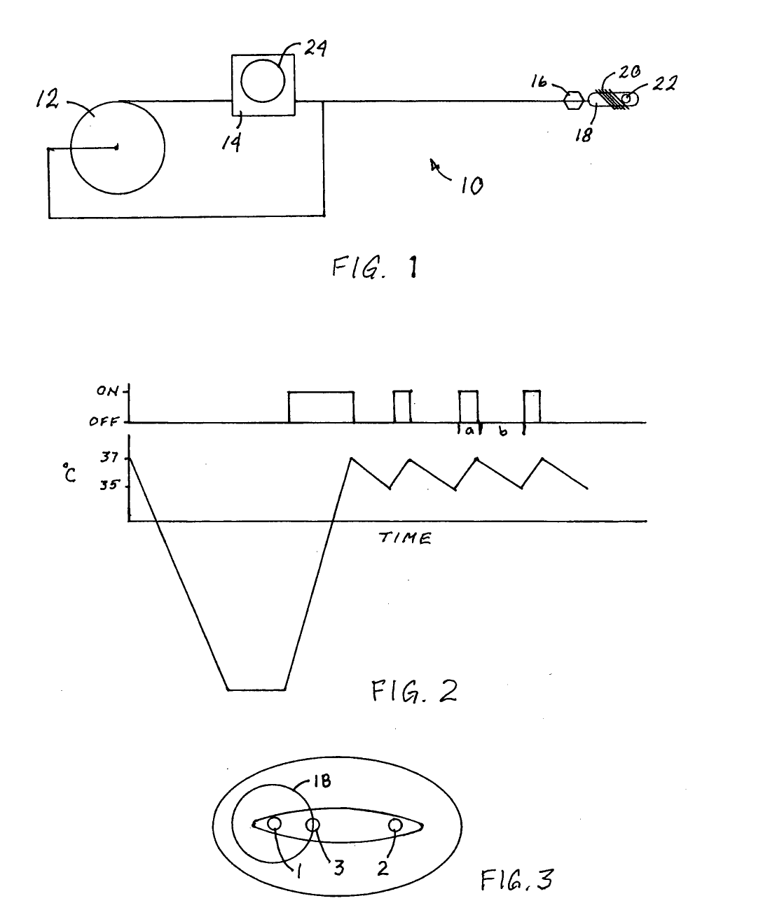

As shown schematically in FIG. 1, the apparatus 10 used in the present invention includes a refrigeration unit 12, a control unit 14 including a processor or group of processors for controlling the operation of the apparatus, a switching mechanism 16 allowing the user to select a freezing mode or a heating mode, a refrigerated probe 18, a heating element 20 and a temperature sensor 22 on the probe 18, and a display unit 24. The heating element 20 can be energized by switching the switch mechanism 16 to the heat mode, to slightly melt a thin layer of ice immediately surrounding the probe 18, to release the probe 18 from the ice ball. The refrigeration unit 12 which cools the probe 18 can continue running while the probe 18 is being heated. Then, when it is desired to form the second ice ball, the heater operation is simply terminated, after which the probe temperature drops, to begin freezing surrounding tissue.

The heating element 20 can be controlled by the control unit 14 to achieve a target probe temperature, as measured by the probe temperature sensor 24, which substantially matches the temperature of the surrounding organ, which is generally about 37 degrees C. An automatic heater control system within the control unit 14 can cyclically apply energy, such as electrical energy, to the heating element 20, to raise the probe 18 to the target temperature and maintain it at the target temperature. That is, application of energy to the heater 20 causes the probe temperature to rise until it reaches the target temperature, such as 37 degrees C., for example. As the probe temperature rises toward the target temperature, a thin layer of the surrounding ice ball will melt, thereby releasing the probe 18 from the ice ball. After the probe 18 reaches the target temperature, the automatic control system will turn the heating element 20 off until the probe 18 reaches a lower limit, such as 35 degrees C., for example. Thereafter, the automatic control system will cyclically turn the heater 20 on and off to maintain the probe 18 substantially at the target temperature, until operation of the heating element 20 is terminated and formation of the second ice ball begins. Use of a wider temperature range could provide less instability due to the change of temperature when the heater 20 is on, and better measurement when the heater 20 is off.

FIG. 2 shows the relationship between the heater on/off states and the probe temperature, when the switching mechanism 16 is in the heating mode. The on/off states are shown in the upper graph as step changes, while the probe temperature in the lower graph is shown as linear slopes corresponding to the rise and fall of the temperature within the temperature range. As used herein, heater “duty cycle” is a calculated value, for a given heating cycle, which represents a comparison between the amount of time that the heater 20 is on, “a” in FIG. 2, and the amount of time that the heater 20 is off, “b” in FIG. 2, or a comparison between the amount of time that the heater 20 is on and the overall period, “a” plus “b” in FIG. 2, of the heating cycle. The comparison of the on/off times can be accomplished by dividing the heater on-time by the heater off-time, or it can be accomplished by dividing the heater on-time by the heater on-time plus off-time. When the probe 18 is re-positioned within the first ice ball, it has been found that the heater duty cycle will be a greater value than when the probe 18 is re-positioned at a location which is at least not fully within the first ice ball. That is, positioning the probe 18 either completely outside the first ice ball or on the edge of the first ice ball will result in a comparatively lower duty cycle, since the probe 18 is not surrounded by frozen tissue which would act as a heat sink. Conversely, positioning the probe 18 fully within the first ice ball will result in a comparatively higher duty cycle, since the probe 18 is fully surrounded by frozen tissue acting as a heat sink.

The present invention makes use of this phenomenon, by monitoring the duty cycle value computed for each successive heater cycle. The user selects a duty cycle setpoint with the control unit 14, based on experience with the particular probe 18 being used and the particular tissue being treated. Best results are usually obtained by selecting a setpoint where the heater duty cycle will maintain probe temperature at the target temperature with the probe 18 positioned in the edge of the “ice ball” of tissue. After several iterations of positioning a particular probe in a particular type of tissue, then observing formation of the second ice ball, the user will quickly arrive at a good duty cycle setpoint to use for future treatments of the same type of tissue with the same probe.

When the heat mode is selected and the duty cycle of an exemplary probe 18 is above the selected setpoint, which can be for example, 20%, the control unit 14 determines that the probe 18 is fully within the first ice ball IB, as shown at position 1 in FIG. 3. In this “in-ice” condition, the control unit 14 gives the user an indication on the display unit 24 that the probe 18 is in ice, and that a second freezing operation should not commence. This type of indication can be referred to as the “in-ice” condition. Conversely, when the duty cycle of an exemplary probe 18 is below the selected setpoint, the control unit 14 determines that the probe 18 is outside the first ice ball, as shown at position 2 in FIG. 3, or at least not fully within the first ice ball, as shown at position 3 in FIG. 3. In either position 2 or position 3, the control unit 14 gives the user an indication on the display unit 24 that the probe 18 is not in ice, and that a second freezing operation can proceed. This type of indication can be referred to as the “proceed” condition.

Selecting a setpoint where the heater duty cycle is expected to maintain probe temperature at the target temperature with the probe 18 positioned in the edge of the “ice ball” of tissue, as at position 3, is preferred over selecting a setpoint where the heater duty cycle is expected to maintain the probe temperature at the target temperature with the probe 18 positioned fully outside the ice ball, as at position 2. This is because, if the setpoint were selected as the duty cycle expected at position 2, a false “in-ice” indication could be caused by proximity to the ice ball. This will result in unnecessary repositioning of the probe 18. On the other hand, if the setpoint is selected as the duty cycle expected at position 3, a “proceed” indication caused by proximity to the warmer tissue will result in beginning a second ice ball on the periphery of the first ice ball. Forming a second ice ball on the periphery of the first ice ball is preferable to the added delay attached to an unnecessary repositioning of the probe 18.

Indications of “in-ice” or “proceed” can be given as either visible or audible indications or both. Alternatively or in combination with visible and audible indications, a numeric indication of duty cycle value can be given to the user, with the significance of the numeric indication being interpreted by the user, based on experience with the particular probe in the particular type of tissue.

It has been found that it can be useful to have a slightly lower setpoint when the probe 18 is warming from a cold temperature than the nominal setpoint used when the probe 18 is cooling from a warmer temperature. This can be accomplished automatically within the control unit 14. For instance, if the selected nominal duty cycle setpoint is 20%, a 2% lower limit of 18% can be automatically set by the control unit 14. Then, if the probe 18 is in ice but the surroundings are warming up, the duty cycle value will progressively diminish. If the duty cycle value diminishes to the lower setpoint of 18%, the indication will change from “in-ice” to “proceed”. On the other hand, if the duty cycle value only diminishes to some value above 18%, such as 19% for example, even though the duty cycle value is less than the nominal setpoint of 20%, the indication will remain “in-ice”. Conversely, if the probe 18 is not in ice but the surroundings are cooling off, the duty cycle value will progressively increase. If the duty cycle value increases to the nominal setpoint of 20%, the indication will change from “proceed” to “in-ice”. On the other hand, if the duty cycle value only increases to some value above 18%, such as 19% for example, the duty cycle value will still be less than the nominal setpoint of 20%, and the indication will remain “proceed”.

It has also been found that after the probe 18 is left in a location for an extended period of time, the surrounding tissue reaches an equilibrium, reducing the reliability of the determination of whether the probe 18 is within the ice ball, and duty cycle value calculation should be terminated. This is because, as the surroundings reach equilibrium, whether in ice or not in ice, either the on-time or the off-time will approach a very long time, causing the duty cycle value to approach 100% or 0%. A time threshold can be selected on the control unit 14 to prevent this eventuality, depending upon the user's experience with the probe 18 and the type of tissue being treated. This time threshold can be on the order of 90 seconds, but it is usually less.

It is desirable for the probe 18 to be relatively stationary before measurement of on-time and off-time, to establish a stable relationship between probe temperature and tissue temperature. So, after the user selects the heat mode, a first time delay, for example 1 second, is implemented before measurements and calculations commence.

A dampening function can be repetitively applied to the duty cycle value, where the dampened duty cycle value equals the sum of the last dampened value times 5, plus the newly calculated duty cycle, with the sum of these divided by 6. After a second time delay after the heat mode is selected, for example 9 seconds, an initial determination is made by the control unit 14 as to whether the probe 18 is in ice or not. The 9 second time period provides sufficient time for the dampening function to be implemented. The dampening function can be further refined by allowing it to vary based on the overall period of the duty cycle. If the period is longer, the dampening can be shorter. If the period is shorter, the dampening can be longer. This refinement can make the “in-ice/proceed” determination more responsive to the actual changes in the duty cycle.

Although this disclosure is made largely in terms of a cryotherapy probe as used in a uterus to treat endometriosis, it should be understood that this invention could have applications to other instruments, or in other organs, or for different types of therapy.

What is claimed:

1. A method for placing an instrument in a treatment zone having an ice ball therein, comprising:
placing an instrument in a desired location within said treatment zone;
maintaining said instrument in a desired temperature range by cyclical application of energy to said instrument;
measuring energy on-time and energy off-time while maintaining said instrument in said desired temperature range, for each successive said energy cycle; and
indicating the location of said instrument relative to said ice ball, based on relative energy on-time and energy off-time for each successive said energy cycle.

2. The method recited in claim 1, wherein said cyclical application of energy comprises cyclically applying heat to maintain said instrument temperature in said desired temperature range.

3. The method recited in claim 1, further comprising:
calculating a duty cycle value for each said energy cycle, from said energy on-time and said energy off-time; and
using said duty cycle value as the basis for said indication of instrument location relative to said ice ball.

4. The method recited in claim 3, wherein said duty cycle is calculated by dividing said on-time by the sum of said on-time plus said off-time.

5. The method recited in claim 3, wherein said duty cycle is calculated by dividing said on-time by said off-time.

6. The method recited in claim 3, wherein said indication of instrument location is made when said duty cycle value is greater than a selected value.

7. The method recited in claim 6, wherein:
said cyclical energy application comprises cyclically applying heat to said instrument; and
said instrument is indicated to be within said ice ball when said duty cycle value is greater than said selected value.

8. The method recited in claim 3, wherein said indication of instrument location is made when said duty cycle value is less than a selected value.

9. The method recited in claim 8, wherein:
said cyclical energy application comprises cyclically applying heat to said instrument; and
said instrument is indicated to be not within said ice ball when said duty cycle value is less than said selected value.

10. A method for placing a cryotherapy probe, comprising:
placing a cryotherapy probe in a first location within a treatment zone;
cooling said probe to form an ice ball in said first location, said ice ball having a temperature lower than the ambient temperature of said treatment zone;
raising the temperature of said probe sufficiently to melt ice immediately adjacent to said probe, thereby releasing said probe from said ice ball;
withdrawing said probe from said ice ball and placing said probe in a second location within said treatment zone;
maintaining said probe in a desired temperature range by cyclical heat application;
measuring heat on-time and heat off-time while maintaining said probe in said desired temperature range, for each successive said heating cycle; and
evaluating relative said heat on-time and said heat off-time for each successive heating cycle to determine whether said probe is within said ice ball.

11. The method recited in claim 10, wherein said evaluating comprises calculating a duty cycle value for each said heating cycle, from said heat on-time and said heat off-time.

12. The method recited in claim 11, further comprising providing an indication that said probe is within said ice ball when said duty cycle value is greater than said selected value.

13. The method recited in claim 11, further comprising providing an indication that said probe is not within said ice ball when said duty cycle value is less than said selected value.

14. The method recited in claim 10, further comprising terminating said evaluation relative to said heat on-time and said heat off-time, when said probe has been in said second location a sufficient time to reduce the reliability of said determination of whether said probe is within said ice ball.

15. A cryotherapy apparatus, comprising:
a cryotherapy probe, said probe having a cooling element and a heating element;
a switching apparatus adapted to selectively maintain said probe in a desired temperature range by cyclical energy application to said heating element;
a temperature sensor adapted to measure the temperature of said probe;
a processor adapted to measure heating element on-time and off-time while maintaining said probe in said desired temperature range, for each successive said heating cycle, said processor being adapted to determine whether or not said probe is within an ice ball, based on relative said heating element on-time and off-time; and
a display device adapted to advise a user as to whether said probe is within said ice ball, based on said determination by said processor.

16. The apparatus recited in claim 15, wherein:
said processor is further adapted to calculate a duty cycle value from said heating element on-time and off-time; and
said processor is further adapted to use said duty cycle value as a basis for indicating whether said probe is within said ice ball, via said display.

17. The apparatus recited in claim 16, wherein said processor is further adapted to calculate said duty cycle by dividing said on-time by the sum of said on-time plus said off-time.

18. The apparatus recited in claim 16, wherein said processor is further adapted to calculate said duty cycle by dividing said on-time by said off-time.

19. The apparatus recited in claim 16, wherein said processor is further adapted to indicate, via said display, that said probe is within said ice ball when said duty cycle value is greater than a selected value.

20. The apparatus recited in claim 16, wherein said processor is further adapted to indicate, via said display, that said probe is not within said ice ball when said duty cycle value is less than a selected value.

21. A method for placing an instrument in a treatment zone having an existing ice ball therein comprising:
placing an instrument in a desired location within said treatment zone; allowing said instrument to automatically pursue a desired temperature while within said treatment zone;
monitoring a temperature-based parameter of said instrument while within said treatment zone and pursuing said desired temperature; and
indicating the location of said instrument relative to said existing ice ball based on the monitored temperature-based parameter of said instrument.

22. The method of claim 21, wherein said step of allowing said instrument to automatically pursue a desired temperature is performed via a cyclical application of energy to said instrument.

23. A method of placing a cryotherapy probe, comprising:
placing a cryotherapy probe in a first location within a treatment zone;
cooling said probe to form an ice ball in said first location, said ice ball having a temperature colder than an ambient temperature of said treatment zone;
raising the temperature of said probe sufficiently to melt ice immediately adjacent to said probe to release said probe from said ice ball;
withdrawing said probe from said ice ball and placing said probe in a second location within said treatment zone;
allowing said probe to automatically pursue a desired temperature while within said second location;
measuring a temperature-based parameter of said probe while within said second location and while pursuing said desired temperature; and
evaluating relative said measured temperature-based parameter whether said probe is within said ice ball.

24. The method of claim 23, wherein said step of allowing said probe to automatically pursue a desired temperature is performed via a cyclical application of energy to said instrument.

25. A cryotherapy apparatus, comprising:
a cryotherapy probe that automatically pursues a desired temperature and produces a temperature-based parameter;
a sensor that monitors said temperature-based parameter and produces a signal representative of said temperature-based parameter;
a processor that receives said signal from said sensor and determines whether said cryotherapy probe is within or proximate an existing ice ball based on said signal from said sensor.

26. The cryotherapy apparatus of claim 25, further comprising a device to advise a user as to whether said probe is within or proximate said existing ice ball based on the determination by said processor.

27. The cryotherapy apparatus of claim 26, wherein said device comprises a display device.

28. The crotherapy apparatus of claim 25, wherein said probe automatically pursues said desired temperature via a cyclical application of energy to said probe.
